(19) United States
(12) Patent Application Publication
(10) Pub. No.: US 2008/0308166 A1
(43) Pub. Date: Dec. 18, 2008

(54) Connector/Device with Reflux Valves

(76) Inventor: Frank M. Richmond, Harvard, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 12/160,698

(22) PCT Filed: Oct. 19, 2006
(86) PCT No.: PCT/US06/40778

§ 371 Date: Jul. 11, 2008

Related U.S. Application Data
(60) Provisional application No. 60/728,479, filed on Oct. 20, 2005.

(51) Int. Cl. A61M 39/24

(52) U.S. Cl. 137/859

(57) ABSTRACT

The present invention provides a device which includes reflux valves in combination with components such that a fluid flow occurs when connections are made and stops when disconnected.

The present invention relates to valved connectors, components and systems. More particularly, connectors are provided that include reflux valves that allow fluid flow when connected and provide a closed connection when disconnected.

### BACKGROUND

Improvements in safety and convenience in handling of fluid compositions are of a continuing interest. Many types of containers, components and systems include connectors or valves which allow those containers to be connected to other components. Commonly used connectors (male and female luers in combination with shut-off clamps or stop-cocks, spikes used in combination with membrane ports, etc.) have not been completely effective for preventing the unwanted escape of fluids from the containers or the entry of contaminants into the containers.

In the medical disposable field, components which are easy to use and which provide improved safety are of particular interest. The trend in the medical disposable field is for containers to have either male or female reflux valves added as a means of access to the containers. Some examples of these types of containers and valves are described in U.S. Pat. Nos. 5,391,150, 5,405,333, 5,645,538, 6,068,617, 6,485,472, and EP0719158. These valve provide ease of use and result in certain safety advantages. However, these valves alone do not provide systems that allow fluid flow when connected and provide a closed connection when disconnected.

### SUMMARY

The present invention provides a connector/device which includes reflux valves which are, or are intended to be in combination with components such that a fluid flow occurs when connections are made and stops when disconnected. The connector/device is effective for preventing accidental spillage of fluids which can result from disconnection of components by restless patients. Further, spillage of fluids is prevented where unintentional disconnects occur where a clamp has not been shut off.

Preferably, a connector/device that includes reflux valve(s) attached to components is provided. The reflux valve are attached to components. A first reflux valve may be connected to or mateable to a second reflux valve. Each reflux valve includes a valve body having a distal end and a proximal end. The valve body defines a fluid passageway providing two-way fluid communication through the body, via one of the distal and proximal ends. A valve member is disposed in the body. The valve member is biased to a first, closed, position where fluid communication is not established. The valve member is moveable, against the bias, to a second, open, position where fluid communication is permitted. A valve element is also disposed in the passageway for movement within the passageway. The valve element includes an engagement surface for moving the valve element to cause the valve member to move to the second position. One or more of these engagement surfaces can be outside of the valve body depending on the valve design.

The resealable device may include one reflux valve that is a male reflux valve and one that is a female reflux valve. Components to which the reflux valve may be attached include medical components which may include containers, fluid lines, tubing, connectors, sets, component(s) parts of components in kits, drip chambers, filters, burette chambers, stopcocks, multiport valves, vials, flow restriction valve, ‘y’-sites and ‘T’-sites, unions, and ambulatory pumps. The reflux valves may also be attached to non-medical components which may include containers, hoses, tires, caulking dispensers, glue dispensers, grease containers, oil containers, brake fluid, soap dispensers and ink cartridges. The reflux valves may be utilized with any medium that can be dispensed through a valve. The resealable device and all associated components can be made from plastics, multi-layered plastics, rubber, metal, powdered metal, glass, paper materials such as cardboard and laminated cardboard or a combination of these materials.

### BRIEF DESCRIPTION OF THE DRAWINGS

FIG. 1 generally illustrates various types of reflux valves which may be attached to a component.

FIG. 2 shows a male and female reflux valve disconnected (FIG. 2A) and connected or engaged (FIG. 2B)

FIG. 3 shows illustrates a male and female reflux valve with non-luer taper connectors which are disconnected (FIG. 3A) and connected or engaged (FIG. 3B)

FIG. 4 illustrates a swabable male and swabable female reflux valve disconnected (FIG. 4A) and connected or engaged (FIG. 4B).

FIG. 5 shows that ends of the male 440 and female 500 reflux valves as shown in FIG. 5A may include any numbers of shapes, such as for example those described in FIG. 5B.

FIG. 6 illustrates a set that includes a container, a female reflux valve on the container, a filter that includes a male reflux valve and a female reflux valve, and a drip chamber.

FIG. 7 shows a system that includes an ambulatory pump with plug, a female reflux valve, a male reflux valve, and on/off or flow control clamp, a delivery rate tube and a male reflux valve.

FIG. 8 shows a resealable/closed container system.

FIG. 9 illustrates reflux valves used in combination with a hose and faucet.

FIG. 10 shows the use of reflux(s) valve for filling of ink cartridges.

FIG. 11 shows a reflux valve on a tire.

### DETAILED DESCRIPTION

The reflux valves and components illustrated in the drawings show various designs. It is understood that any person skilled in the art can vary the reflux valve configurations and components to which the reflux valve is attached in a number of different ways, within the scope of the invention as defined by the appended claims. A reflux valve(s) and one of various components may be attached to each other using methods known in the art such as for example, solvent bonding, RF welding, ultrasonic welding, spin welding, insertion molded, crimping, gluing, soldering, resistance welding, fusion welding, and other means known in the art. The reflux valves and components may be completely or in part be made out of materials known in the art, such as for example, plastics, multi-layered plastics, rubber, metal, powdered metal, glass, paper materials such as cardboard and laminated cardborad, and any other known materials or any combination of these materials.

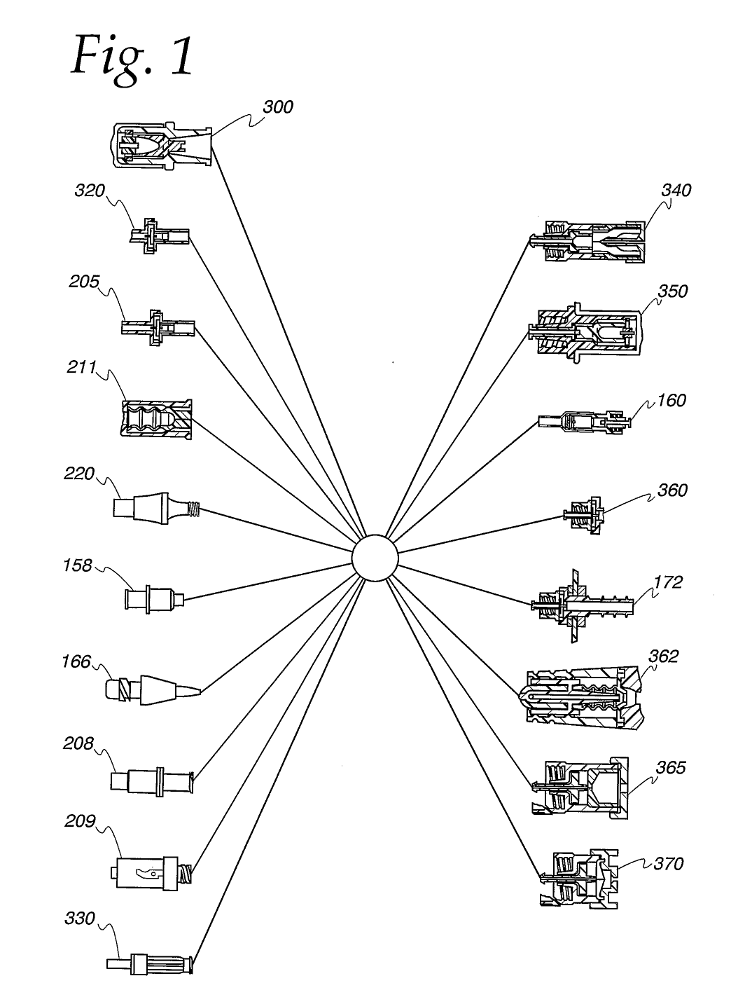

As generally shown in FIG. 1, reflux valves that my be attached to a component may include any number of configurations. Some examples of female reflux valves include Halkey Roberts female reflux valve 300, B. Braun female reflux valve 320, female reflux valve 205 that includes a male luer 102, Alaris swabable female reflux valve 211, ICU swabable female reflux valve 220, Halkey Roberts female valve 158, B. Braun swabable female reflux valve 166, Filtertech swabable female reflux valve 208, Porex swabable female reflux valve 209, and B. Braun split septum swabable female reflux valve 330. Any female reflux valve may be utilized, including for example, female reflux valves available from B. Braun, Alaris, Haulkey-Roberts, Filtertec, Bola, Porex, and other known manufacturers.

Some examples of male reflux valves include a male reflux valve with split septum 340, male reflux valve 350, male reflux valve 160, male reflux valve with disk design 360, male reflux valve with a bulkhead fitting configuration 172, swabable male reflux valve 362, and male reflux valves 365 and 370. The female and male reflux valve may include any of those described in U.S. Pat. Nos. 6,485,472, 6,206,860, 6,106,502, 6,068,617, 5,848,994, 5,735,826, 5,645,538, 5,476,449, 5,445,630, 5,445,623, 5,405,333, 5,391,150, 5,298,024, 5,022,538 and EP0719158 which are incorporated herein by reference.

One example of valves that may be utilized includes valves that include a resilient valve member. In this aspect, the resilient valve member defines an outer periphery that is uninterrupted within the periphery. The valve member is deformable to a configuration wherein fluid communication is permitted. In another related aspect, the valve may include a valve member defining an outer periphery that is interrupted at least once within the periphery. The interruption within the periphery allows the fluid to pass directly through the member.

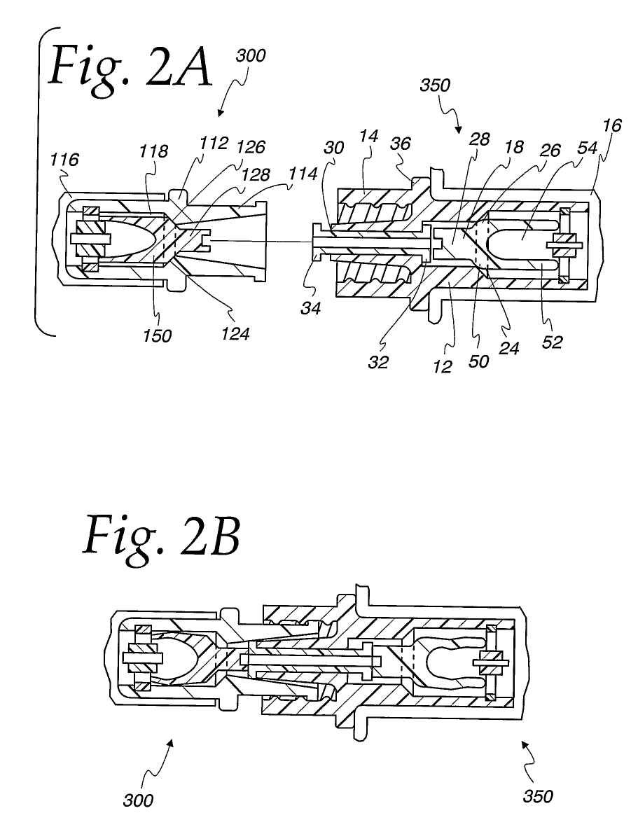

FIG. 2 illustrates a male 350 and female 300 reflux valve disconnected (FIG. 2A) and connected or engaged (FIG. 2B). As shown in FIG. 2A, the male reflux valve 350 includes a valve body 12 having a first end 14 configured like a male luer fitting and a second end 16 which is attached to a component. A fluid passageway 18 is established through the valve body 12, from end 14 to end 16. As those skilled in the art will recognize, male luer fittings need not have the threaded cylindrical outer flange shown in FIG. 2. The male luer fitting may have no means of retention or may have other means of attachment known in the art such as a bayonet connection, snap connection, goal-post connection, spring loaded ball connection, etc.

To selectively block the fluid passageway 18, a valve member 50 is disposed therein. Also, the valve body 12 is formed in this particular embodiment with a frusto-conical seat 26, and as shown in FIG. 2A the valve member 50 is materially biased to a closed configuration, wherein the shoulder 24 flushly contacts the seat 26 to thereby block the fluid passageway 18. Moreover, pressure can be exerted against a stem 28 of the valve member 50 to move the valve member 50 to an open configuration, such that the shoulder 24 is distanced from the seat 26. In the open configuration, fluid can pass between the shoulder 24 and seat 26, and the fluid passageway 18 consequently is unblocked.

FIG. 2A additionally shows in this embodiment that a valve element 30 is disposed in the fluid passageway 18. The valve element 30 is formed with a lower contact flange 32 and an upper contact flange 34. As shown, the lower contact flange 32 retains the valve element 30 in the valve body 12 by contacting a flange surface 36.

FIG. 2A further illustrates a female reflux valve 300. The female reflux valve 300 includes a valve body 112 having a first end 114 configured like a female luer fitting and a second end 116 which is attached to a component. A fluid passageway 118 is established through the valve body 112, from end 114 to end 116.

To selectively block the fluid passageway 118, a valve member 150 is disposed therein. Also, the valve body 112 in this embodiment is formed with a frusto-conical seat 126, and as shown in FIG. 2A, the valve member 150 is materially biased to a closed configuration, wherein the shoulder 124 flushly contacts the seat 126 to thereby block the fluid passageway 118. Moreover, pressure can be exerted against a stem 128 of the valve member 150 to move the valve member 150 away from seat 126 against the bias acting on the valve member 150 to move the valve member 150 to an open configuration, such that the shoulder 124 is distanced from the seat 126. In the open configuration, fluid can pass between the shoulder 124 and seat 126, and the fluid passageway 18 consequently is unblocked.

As shown in FIG. 2B, it can now be understood that the male luer fitting-configured end 14 of the valve 350 can be engaged with a complementarily-shaped female luer fitting 300, and that by so engaging the valve 350, the flange 34 of the valve element 30 is contacted by the stem 128 of the female luer fitting 300 to urge the valve element 30 downwardly. When the valve element 30 is urged sufficiently downwardly, it in turn contacts the valve member 50 and urges the valve member 50 to the open configuration, to thereby unblock the fluid passageway 18. Correspondingly, the stem 128 of the female reflux valve 300 urges the valve member 150 to move to an open configuration, to unblock fluid passageway 118.

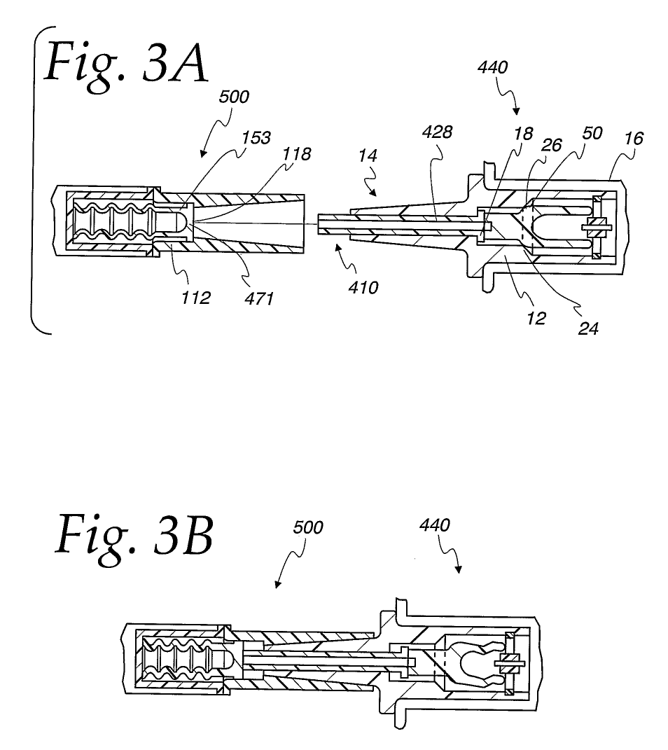

FIG. 3 illustrates a male 440 and female 500 reflux valve with non-luer taper connectors which are disconnected (FIG. 3A) and connected or engaged (FIG. 3B). These reflux valve are of similar construction to those described in detail with reference to FIGS. 2A and 2B and will be further describes except to the extend they differ. As shown in FIG. 3A, the male reflux valve 440 includes a valve body 12 having a first end 14 configured like as male tapper connector or fitting 410 and a second end 16 which is attached to a component. A fluid passageway 18 is established through the valve body 12, from end 14 to end 16.

To selectively block the fluid passageway 118 of the female reflux valve 500, a valve member 153 is disposed therein. The valve member 153 is materially biased to a closed configuration thereby blocking the fluid passageway 118. Moreover, pressure can be exerted against valve member 153 to move the valve member 153 to an open configuration of the female reflux valve, such that a slit 471 is opened and fluid can pass through and/or around slit 471.

As shown in FIG. 3B, it can now be understood that the male end 410 of the valve 440 can be engaged with a complementarily-shaped female fitting of female reflux valve 500. By so engaging the male valve 440, the valve member 153 is contacted by the male tapper end 410. When the valve member 153 is sufficiently urged, slit 471 opens to thereby unblock the fluid passageway 118. Correspondingly, is exerted against a stem 428 of the valve member 50 to move the valve member 50 to an open configuration, such that the shoulder 24 is distanced from the seat 26. In this open configuration of the male reflux valve 440, fluid can pass between the shoulder 24 and seat 26, and the fluid passageway 18 consequently is unblocked.

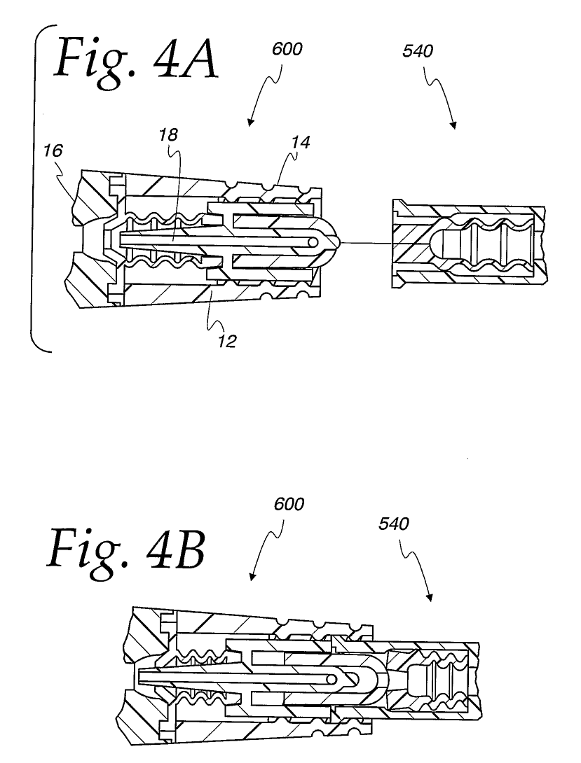

FIG. 4 illustrates a swabable male 600 and swabable female reflux valve 540 disconnected (FIG. 4A) and connected or engaged (FIG. 4B). As shown in FIG. 4A, the swabable male reflux valve 600 includes a valve body 12 having a first end 14 configured like a male luer fitting and a second end 16 which is attached to a component. A fluid passageway 18 is established through the valve body 12, from end 14 to end 16. As those skilled in the art will recognize, male luer fittings need not have the threaded cylindrical outer flange shown in FIG. 4. The male luer fitting may have no means of retention, or it can have any other means of attachment known in the art such a bayonet connection, snap connection, goal-post connection, spring loaded ball connection, etc. As in the preceding embodiments, the male and reflux valves are closed when the two valves are kept separate, but they are both open when the two valves are mated together.

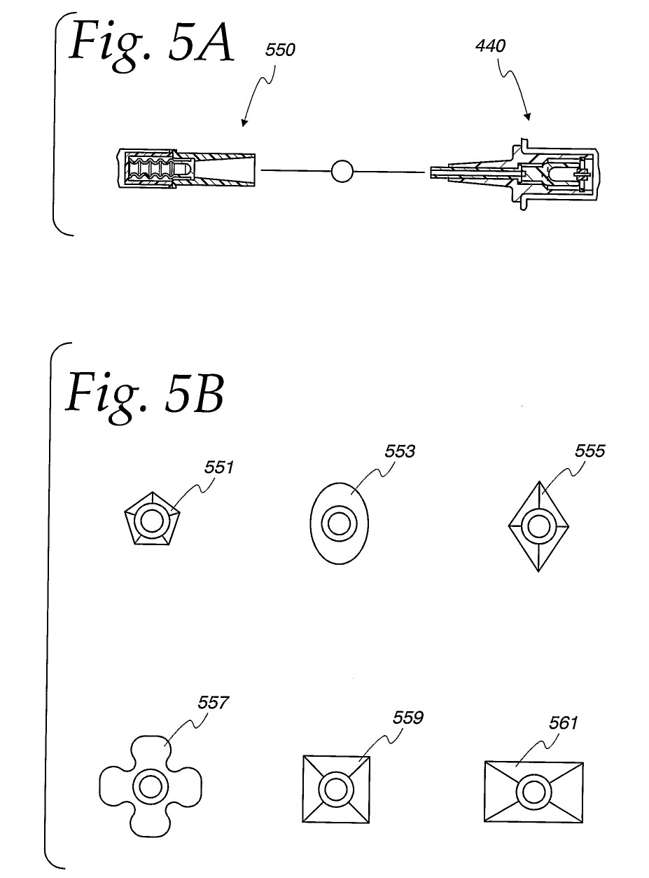

FIG. 5 illustrates that ends of the male 440 and female 500 reflux valves as shown in FIG. 5A may include any numbers of cross-sectional shapes, such as for example those described in FIG. 5B. End shapes which may be used include multiside 551, oval 553, diamond 555, clover leaf 557, square 559, and oblong 561, etc.

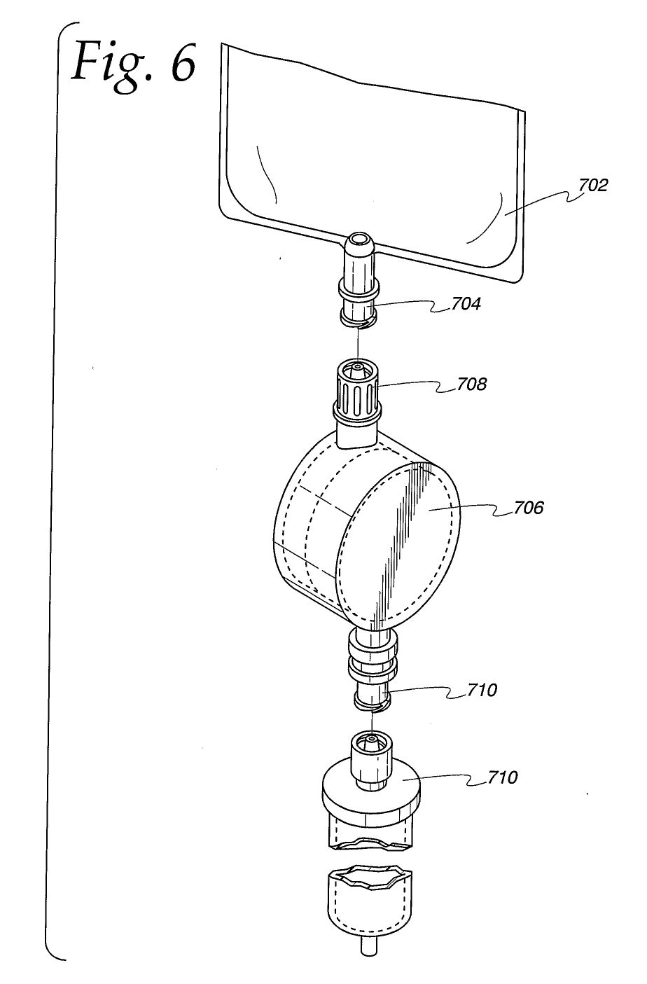

FIG. 6 illustrates a set 700 that includes a container 702, a female reflux valve 704 on the container 702, a filter 706 that includes a male reflux valve 708 and a female reflux valve 710, and a drip chamber 711. The drip chamber 711 includes a male reflux valve (not shown). The male/female valve parts 704/708 and 710 plus the unillustrated male reflux valve each have intermateable male and female reflux valves such as described in detail in the preceding embodiments.

FIG. 7 shows a system that includes an ambulatory pump 712 with plug 714, intermateable female reflux valve 704 and male reflux valve 708, and on/off or flow control clamp 715, a delivery rate tube 716 and a male reflux valve 708.

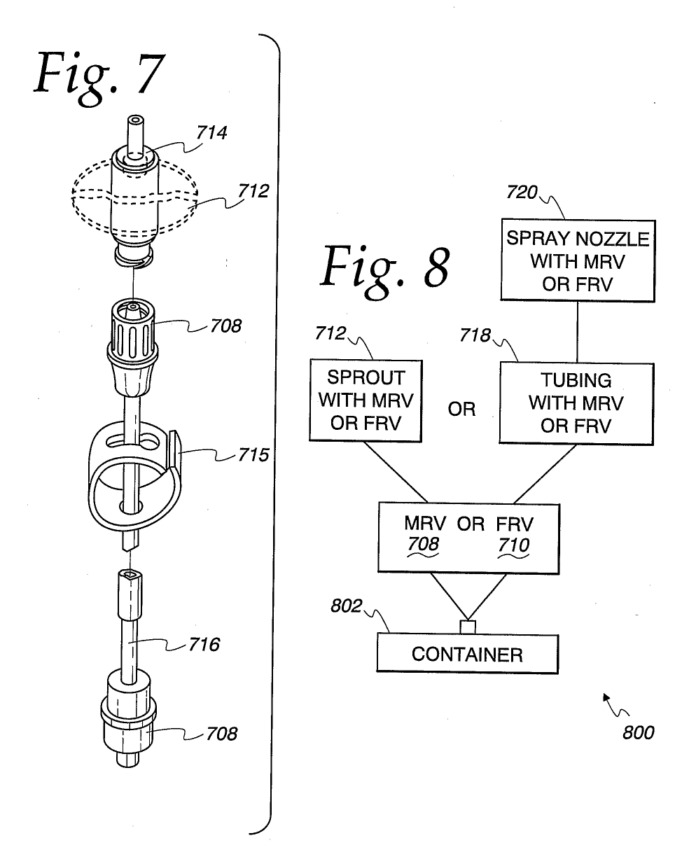

FIG. 8 illustrates a closed/resealable connector system 800. The system includes a container 802. The container 802 may be a made of known materials such as plastic, glass, rubber, cardboard, laminated cardboard, powdered metal, metal or combinations thereof. The container may be utilized for example for compositions such as soap, cosmetics, food items, oil, brake fluid, or gas. The container 802 may include a male reflux valve 708 or female reflux valve 710. The male 708 or female reflux valve 710 may be connected to another female or male reflux valve which is attached to another component. For example, the other component may be a spout 712 with a female male reflux valve or a tubing 718 with a female or male reflux valve. The tubing 718 may include a reflux valve on either end and may further attach to another component such as for example, a spray nozzle 720, in corresponding fashion.

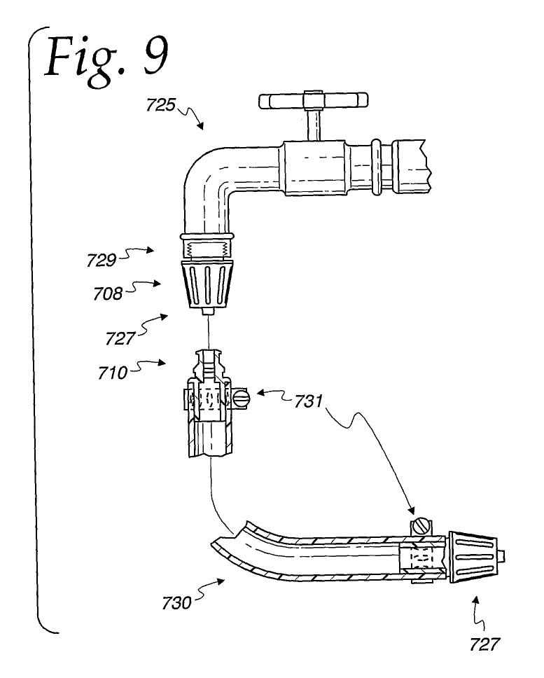

FIG. 9 shows reflux valves used in combination with a hose 730 and faucet 725. A male reflux valve 708, or alternatively a female reflux valve (not shown) is attached to a faucet 725. The reflux valve 708 may include a rotating collar 727 and may be attached to the faucet with screw threads 729. A hose 730 is provided at a fist end with a female reflux valve 710 or alternatively a male reflux valve. The opposite end of the hose may also include a male 708 or female reflux valve. The reflux valves may be attached to hose 730 by any means known in the art, such as for example a hose clamp 731, crimping or collar (not shown).

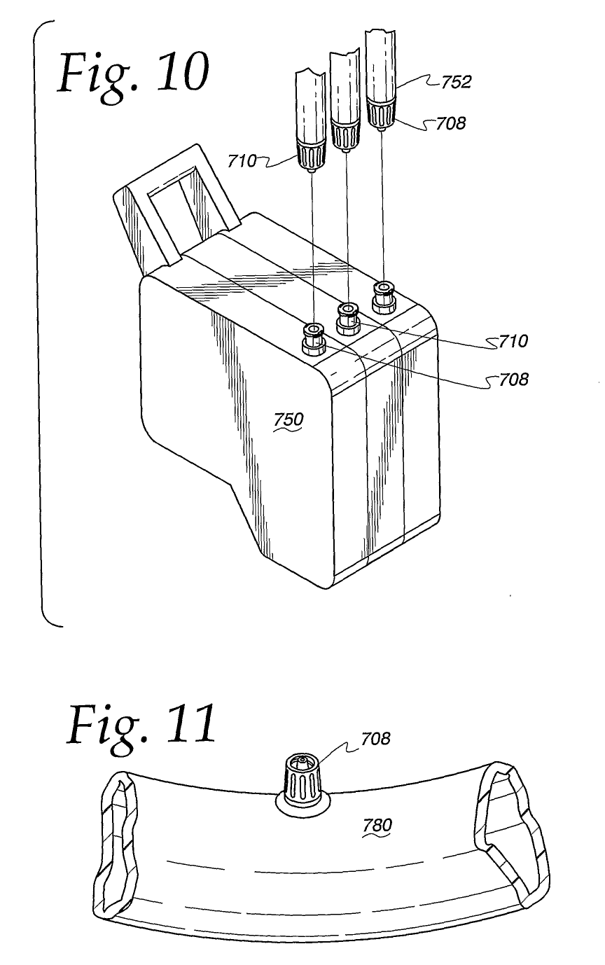

FIG. 10 illustrates the use of reflux valve for filling of ink cartridges, especially ink cartridges used in printers. As shown in FIG. 10, an ink cartridge 750 may include any number of male 708 or female reflux valves 710. The reflux valve on the ink cartridge 750 may all be male, female or a combination of the two. A dispenser 752 is provided that has connecting male 708 or female 710 reflux valve. The dispenser may have one or the same number of male or female reflux valves as the ink cartridge 750 so that the ink can be dispensed to the cartridge. The connection through interacting male/female valve(s) from the dispenser to the cartridge allows ink to flow into the cartridge. Disconnection of the valves stops the flow of ink, seals both the cartridge and the dispensr, and prevents ink from leaking from the dispenser and the cartridge.

FIG. 11 shows a reflux valve on a tire 780. The tire may include a male reflux valve 708 as shown, but alternatively include a female reflux valve. An opposite male or female reflux valve attached to an air compressor hose or pressure valve may be easily attached to the reflux valve on the tire 780. Connection of the valve allows for air flow and when the valves are disconnected air flow is stopped.

The “components” to which reflux valves may be attached include for example medical components, containers, fluid lines, tubing, connectors, syringe, sets, kits, drip chambers, filters, burette chambers, stopcocks, multiport valves, vials, flow restriction valve, ‘Y’-sites and ‘T’-sites, sets (for example gravity sets, pump sets, specialty sets and the like), and ambulatory pumps, or valves of any design. Kits may include all or most of the various components needed for a given procedure. Connectors may be one or more of the components in a kit.

Numerous modifications and variations in practice of the invention are expected to occur to those skilled in the art upon consideration of the foregoing detailed description of the invention. The reflux valves and components described show various designs. It is understood that any person skilled in the art can vary the reflux valve configurations and components to which the reflux valve is attached in a number of different ways, within the scope of the invention as defined by the appended claims. Consequently, such modifications and variations are intended to be included within the scope of the following claims.

What is claimed is:

1. A connector/device comprising a first reflux valve and a second reflux valve, the first reflux valve being mateable to the second reflux valve and each reflux valve comprising
a valve body having a distal end and a proximal end, the body defining a fluid passageway providing two-way fluid communication through the body, via one of the distal and proximal ends, and
a valve member disposed in the body, the valve member being biased to a first, closed, position wherein said fluid communication is not established, the member being moveable to a second, open, position wherein said fluid communication is permitted;
each valve member having a valve element disposed in the passageway for displacement within the passageway, such that, with the first reflux valve mated to the second reflux valve, each valve element displaces the other one to cause both valve members to move to their respective second positions, thereby providing fluid communication through the connector device.

2. The device of claim 1 wherein one reflux valve is a male reflux valve and one is a female reflux valve.

3. The device of claim 1 or two wherein each reflux valve is attached to a separate component, connector, device and/or system so that fluid communication is established between the components when the first reflux valve is mated to the second reflux valve.

4. The device of claim 3 wherein the component is a medical component selected from the group consisting of containers, fluid lines, syringes, tubing, connectors, sets, kits, drip chambers, filters, burette chambers, stopcocks, multiport valves, vials, flow restriction valve, ‘Y’-sites and ‘T’-sites, unions, ambulatory pumps, and combinations thereof.

5. The device of claim 3 wherein the component is a component selected from the group consisting of containers, hoses, tires, ink cartridges, caulking cartridges, tubular containers, glue cartridges, grease cartridges, metal cans, media dispensers, and combinations thereof.
